(19) United States
(12) Patent Application Publication
(10) Pub. No.: US 2012/0156256 A1
(43) Pub. Date: Jun. 21, 2012

(54) NANOPARTICLES HAVING AT LEAST ONE ACTIVE INGREDIENT AND AT LEAST TWO POLYELECTROLYTES

(75) Inventors: Cecile Bonnet-Gonnet, Lyon (FR); Rémi Meyrueix, Lyon (FR)

(73) Assignee: Flamel Technologies, Venissieux (FR)

(21) Appl. No.: 13/328,696

(22) Filed: Dec. 16, 2011

Related U.S. Application Data
(60) Provisional application No. 61/424,280, filed on Dec. 17, 2010.

(30) Foreign Application Priority Data
Dec. 17, 2010 (FR) 10 60684
Dec. 16, 2011 (IB) PCT/IB2011/055728

(51) Int. Cl. A61K 9/00; B32B 5/16; G01N 37/00; B82Y 5/00

(52) U.S. Cl. 424/400; 252/408.1; 428/402; 977/773; 977/906; 977/915; 977/926

(57) ABSTRACT

The present invention relates to novel nanoparticles formed by at least one active ingredient and by at least two polyelectrolytes of opposite polarity, in particular characterized in that at least one of the two polyelectrolytes bears hydrophobic side groups and at least one of the two polyelectrolytes bears side groups of the polyalkylene glycol type, said nanoparticles having an average diameter ranging from 10 to 100 nm and comprising a quantity of groups of the polyalkylene glycol type such that the mass ratio w_{PAG} of polyalkylene glycol relative to the total polymer is greater than or equal to 0.05.

The invention relates to novel nanoparticles formed by at least two specific polyelectrolytes of opposite polarity and by at least one active ingredient, and the formulations comprising said nanoparticles.

The formulations of active ingredient must comply with a certain number of tolerance criteria, have a sufficient concentration of active ingredient, while having a low viscosity in order to allow easy injection through a needle with a small diameter, for example a 27- to 31-gauge needle.

In this field, the applicant company has succeeded in developing, as presented in document WO 2008/135561, stable suspensions with low viscosity, constituted by microparticles loaded with active ingredient. These microparticles, capable of releasing the active ingredient over an extended period, are more particularly formed from the mixture, under specific conditions, of two polyelectrolyte polymers (PE1) and (PE2) of opposite polarity, at least one of which bears hydrophobic groups. This mixture leads to microparticles of a size comprised between 1 and 100 μm.

However, the formulations of microparticles are not suitable for intravenous administration and may, on the occasion of administration by subcutaneous route, pose problems of intolerance.

Consequently, from the viewpoint of administration of active ingredients by parenteral, in particular intravenous or subcutaneous, route, it would be preferable to have suspensions of particles of even smaller size, and in particular of nanometric scale.

Moreover, the subcutaneous administration of active ingredients requires the volume of the dose injected to be limited, for example less than or equal to 1 mL, and consequently requires the formulation of active ingredient to be sufficiently concentrated. This constraint is particularly limiting for peptides or certain small molecules, the therapeutic doses of which are generally high.

Besides, obtaining a concentrated suspension of particles of active ingredient, from a dilute suspension, is restrictive, in particular requiring the application of one or more stages of concentration, to result in a dose that can be administered to the patient.

Therefore there is still a need for stable formulations of nanoparticles of active ingredient, sufficiently concentrated, and nevertheless having a low viscosity, particularly suitable for an administration by parenteral, in particular intravenous, route.

The present invention specifically aims to propose novel nanoparticles, and novel compositions containing the latter, which are able to meet all of the above-mentioned requirements.

Against all expectations, the inventors have discovered that it is possible to obtain concentrated fluid formulations of nanoparticles loaded with active ingredient, from a mixture of specific polyelectrolytes.

More precisely, according to a first of its aspects, the present invention relates to nanoparticles formed by at least one active ingredient and by at least two polyelectrolytes of opposite polarity having a linear backbone of the polyamino acid type and having a degree of polymerization less than or equal to 2,000, characterized in that:
at least one of the two polyelectrolytes bears hydrophobic side groups;
at least one of the two polyelectrolytes bears side groups of the polyalkylene glycol type;
   said nanoparticles having an average diameter ranging from 10 to 100 nm and comprising a quantity of groups of the polyalkylene glycol type such that the mass ratio w_{PAG} of polyalkylene glycol relative to the total polymer is greater than or equal to 0.05.

In particular, the mass ratio w_{PAG} of polyalkylene glycol relative to the total polymer ranges from 0.1 to 0.75, in particular from 0.15 to 0.6, in particular from 0.15 to 0.5 and preferably from 0.15 to 0.3.

Advantageously, the polyelectrolytes considered according to the invention are biocompatible. They are perfectly tolerated and degrade rapidly, i.e. on a time scale of a few days to a few weeks.

According to another of its aspects, the invention relates to a composition, in particular pharmaceutical, comprising at least nanoparticles as defined previously.

In particular, the nanoparticles according to the invention prove to be particularly advantageous as vehicles for protein and peptide active ingredients, and/or for solubilizing active ingredients of low molecular mass.

Besides, the nanoparticles according to the invention are advantageously capable of releasing the active ingredient over an extended period.

The nanometric size of the particles of the invention is moreover particularly suited to administration of the formulation of active ingredients by intravenous or subcutaneous route. The present invention thus proves to be particularly advantageous with regard to the parenteral administration of active ingredients used for the treatment of cancers.

Various formulations of polyelectrolytes have already been described.

Thus, Kabanov et al., Macromolecules, 1996, 29, 6797-6802, describe nanoparticles formed by complexation of two polyelectrolytes of opposite polarity, and more precisely of the diblock poly(sodium methacrylate)-b-PEO as anionic polyelectrolyte and poly(N-ethyl-4-vinylpyrimidium bromide) as cationic polyelectrolyte. However, it may be difficult to envisage parenteral administration of non-biodegradable polyelectrolytes of this type.

Kataoka et al., in the documents Lee Y. and Kataoka K., Soft Matter, 2009, 5, 3810-17 and Osada K. et al., J.R. Soc. Interface, 2009, 6, S325-S339, describe polyionic micelles, in particular formed by polyethylene glycol)-polyamino acid block copolymers, for parenteral administration of active ingredients, more particularly of anti-cancer active ingredients such as doxorubicin.

Sonaje et al., Biomaterials, 2010, 31, 3384-3394, describe polyelectrolyte complexes obtained by complexation of chitosan with p-gamma glutamic acid, combining insulin.

However, as far as the inventors are aware, nanoparticles combining two polyelectrolytes complying with the abovementioned specific requirements of the present invention have never been proposed.

According to another of its aspects, the present invention relates to a method for the preparation of nanoparticles having an average diameter ranging from 10 to 100 nm, characterized in that it comprises at least the stages consisting of:

(1) having an aqueous solution comprising nanoparticles of a first polyelectrolyte in the charged state, bearing hydrophobic side groups, said nanoparticles being non-covalently combined with an active ingredient;

(2) bringing said solution (1) together with at least one second polyelectrolyte of opposite polarity to that of the first polyelectrolyte, so as to form said nanoparticles,

with at least one of said first and second polyelectrolytes having side groups of the polyalkylene glycol type, the quantity of said groups of the polyalkylene glycol type being such that the mass ratio w_{PAG} of polyalkylene glycol relative to the total polymer is greater than or equal to 0.05,

said first and second polyelectrolytes having a linear backbone of the polyamino acid type and having a degree of polymerization less than or equal to 2,000.

In particular, the aqueous solution (1) is obtained by adding the active ingredient to an aqueous colloidal solution of the first polyelectrolyte, said active ingredient combining non-covalently with the nanoparticles of said first polyelectrolyte.

The formulations of nanoparticles of active ingredient according to the invention also prove to be particularly advantageous in several respects.

Firstly, a suspension of nanoparticles according to the invention advantageously has an excellent stability. Mixing can moreover be carried out at high concentrations without impairing the physicochemical properties of the suspension, in particular in terms of viscosity, particle size, colloidal or chemical stability. It is thus possible according to the invention to obtain a stable suspension of nanoparticles that is fluid and sufficiently concentrated. In particular, the suspension obtained according to the invention does not require application of a subsequent stage of concentration. The present invention therefore makes it possible to formulate a fluid suspension that is “ready to use”, in particular for administration by intravenous route. In other words, it can be suitable for administration to the patient in its form as obtained at the end of the above-mentioned method.

In addition, a suspension of the nanoparticles according to the invention readily lends itself to lyophilization and to reconstitution in aqueous phase, without affecting the properties obtained.

Moreover, the suspension of nanoparticles according to the invention can be formed extemporaneously at the time of administration by simply mixing two liquid suspensions prepared as described above. Thus, these suspensions of nanoparticles can easily be stored, allowing a limited production cost on the industrial scale to be envisaged.

Finally, the active ingredient is used in an aqueous method not requiring excessive temperature, significant shearing, surfactant, or organic solvent, which advantageously makes it possible to avoid any potential degradation of the active ingredient. Such a characteristic appears to be particularly advantageous with regard to certain active ingredients, such as peptides and proteins, which can potentially be degraded when they are subjected to the abovementioned conditions.

Active Ingredients

Regarding the active ingredient, it can be a molecule of therapeutic, cosmetic or prophylactic interest or of interest for imaging.

It is preferably chosen from the group comprising: proteins, glycoproteins, proteins covalently bound to one or more polyalkylene glycol chains [preferably polyethylene glycol (PEG)], peptides, polysaccharides, liposaccharides, oligonucleotides, polynucleotides, synthetic pharmaceutical substances and mixtures thereof.

More preferably, the active ingredient is chosen from the subgroup comprising erythropoietins, haemoglobin raffimer, analogues or derivatives thereof; oxytocin, vasopressin, adrenocorticotropic hormone, growth factors, blood factors, haemoglobin, cytochromes, the albumins prolactin, luliberin (luteinizing hormone releasing hormone or LHRH) or analogues such as leuprolide, goserelin, triptorelin, buserelin, nafarelin; LHRH antagonists, LHRH competitors, human, porcine or bovine growth hormones (GH), growth hormone releasing hormone, insulin, somatostatin, glucagon, interleukins or mixtures thereof, interferons such as interferon: alpha, alpha-2b, beta, beta-1a, or gamma; gastrin, tetragastrin, pentagastrin, urogastrone, secretin, calcitonin, enkephalins, endomorphins, angiotensins, thyrotropin-releasing factor (TRF), tumour necrosis factor (TNF), nerve growth factor (NGF), growth factors such as beclapermin, trafermin, ancestim, keratinocyte growth factor, granulocyte-colony stimulating factor (G-CSF), granulocyte-macrophage-colony stimulating factor (GM-CSF), macrophage-colony stimulating factor (M-CSF), heparinase, bone morphogenetic protein (BMP), hANP, glucagon-like peptide (GLP-I), VEG-F, recombinant hepatitis B antigen (rHBsAg), renin, cytokines, cyclosporines and synthetic analogues, pharmaceutically active modifications and fragments of enzymes, of cytokines, of antibodies, of antigens and of vaccines, antibodies such as rituximab, infliximab, trastuzumab, adalimumab, omalizumab, tositumomab, efalizumab, and cetuximab.

Other active ingredients are polysaccharides (for example heparin) and oligo- or polynucleotides, DNA, RNA, iRNA, antibiotics and living cells, risperidone, zuclopenthixol, fluphenazine, perphenazine, flupentixol, haloperidol, fluspirilene, quetiapine, clozapine, amisulpride, sulpiride, ziprasidone, etc.

More particularly, the active ingredient is chosen from growth hormone, insulin, calcitonin and cytokines.

Polyelectrolytes

As previously stated, the nanoparticles according to the invention comprise at least two polyelectrolytes of opposite polarity. In other words, the nanoparticles according to the invention comprise at least one anionic polyelectrolyte and at least one cationic polyelectrolyte.

By “polyelectrolyte” is meant, within the meaning of the present invention, a polymer bearing groups capable of ionizing in water, in particular at a pH ranging from 5 to 8, which creates a charge on the polymer. Thus, in solution in a polar solvent such as water, a polyelectrolyte dissociates, causing charges to appear on its backbone and counter-ions in solution.

The polyelectrolytes according to the invention can comprise a set of identical or different electrolyte groups.

Unless otherwise specified, the polyelectrolytes are described, throughout the remainder of the description, as they appear at the pH value of a mixture of the anionic and cationic polyelectrolytes leading to formation of the nanoparticles according to the invention. The description of a group as “cationic” or as “anionic” is considered for example in the light of the charge borne by this group at this pH value of a mixture of the anionic and cationic polyelectrolytes. Similarly, the polarity of a polyelectrolyte is defined in the light of the overall charge borne by this polyelectrolyte at this pH, value.

In particular, the pH value of a mixture of the anionic and cationic polyelectrolytes leading to formation of the nanoparticles according to the invention ranges from 5 to 8, preferably from 6 to 7.5.

More particularly, by “anionic polyelectrolyte” is meant a polyelectrolyte having a negative overall charge at the pH value of a mixture of the two polyelectrolytes.

Similarly, by “cationic polyelectrolyte” is meant a polyelectrolyte having a positive overall charge at the value of a mixture of the two polyelectrolytes.

By “overall charge” of a polyelectrolyte is meant the algebraic sum of all of the positive and negative charges borne by this polyelectrolyte.

Linear Backbone of the Polyamino Acid Type

As mentioned previously; the polyelectrolytes considered according to the invention have a linear backbone of the polyamino acid type, i.e. comprising amino acid residues.

Advantageously, the polyelectrolytes according to the invention are biodegradable.

Within the meaning of the invention, the term “polyamino acid” covers both natural polyamino acids and synthetic polyamino acids.

The polyamino acids are linear polymers, advantageously composed of alpha-amino acids linked by peptide bonds.

There are numerous synthesis techniques for forming block or random polymers, multiple-chain polymers and polymers containing a particular sequence of amino acids (cf. Encyclopedia of Polymer Science and Engineering, volume 12, page 786; John Wiley & Sons).

A person skilled in the art is capable, by virtue of their knowledge, of implementing these techniques in order to obtain polymers suitable for the invention. In particular, reference can also be made to the teaching of documents WO 96/29991, WO 03/104303, WO 2006/079614 and WO 2008/135563.

According to a preferred embodiment variant, the polyamino acid chain is constituted by a homopolymer of alpha-L-glutamate or of alpha-L-glutamic acid.

According to another embodiment variant, the polyamino acid chain is constituted by a homopolymer of alpha-L-aspartate or of alpha-L-aspartic acid.

According to another embodiment variant, the polyamino acid chain is constituted by a copolymer of alpha-L-aspartate/alpha-L-glutamate or of alpha-L-aspartic/alpha-L-glutamic acid.

Such polyamino acids are in particular described in documents WO 03/104303, WO 2006/079614 and WO 2008/135563, the contents of which are incorporated by way of reference. These polyamino acids can also be of the type of those described in Patent Application WO 00/30618.

These polymers can be obtained by methods known to a person skilled in the art.

A certain number of polymers which can be used according to the invention, for example of the poly(alpha-L-glutamic acid), poly(alpha-D-glutamic acid), poly(alpha-D,L-glutamate) and poly(gamma-L-glutamic acid) type of variable masses are commercially available.

Poly(L-glutamic acid) can also be synthesized according to the route described in Patent Application FR 2 801 226.

According to a particularly advantageous embodiment, the anionic polyelectrolyte considered according to the invention is of the following formula (I) or one of its pharmaceutically acceptable salts,

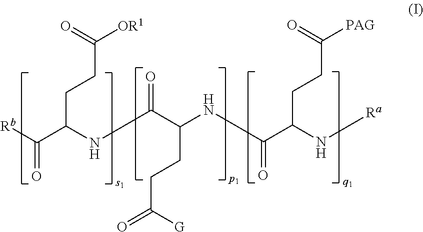

in which:
R^{a} represents a hydrogen atom, a linear C₂ to C₁₀ acyl group, a branched C₃ to C₁₀ acyl group, a pyroglutamate group or a hydrophobic group G as defined below;
R^{b} represents an —NHR⁵ group or a terminal amino acid residue bound by nitrogen and the carboxyl of which is optionally substituted by an —NHR⁵ alkylamino radical or an —OR⁶ alkoxy, in which:
   R⁵ represents a hydrogen atom, a linear C₁ to C₁₀ alkyl group, a branched C₃ to C₁₀ alkyl group, or a benzyl group;
   R⁶ represents a hydrogen atom, a linear C₁ to C₁₀ alkyl group, a branched C₃ to C₁₀ alkyl group, a benzyl group or a group G;
R¹ represents a hydrogen atom or a monovalent metal cation, preferably a sodium or potassium ion,
G represents a hydrophobic group chosen from: octyloxy-, dodecyloxy-, tetradecyloxy-, hexadecyloxy-, octadecyloxy-, 9-octadecenyloxy-, tocopheryl- and cholesteryl-;
PAG represents a polyalkylene glycol, preferably having a molar mass ranging from 1,800 to 6,000 g/mol, in particular a polyethylene glycol, in particular of molar mass ranging from 2,000 to 6,000 g/mol,
   s₁ corresponds to the average number of non-grafted glutamate monomers, anionic at neutral pH,
   p₁ corresponds to the average number of glutamate monomers bearing a hydrophobic group G, and
   q₁ corresponds to the average number of glutamate monomers bearing a polyalkylene glycol group,

p₁ and q₁ optionally being zero,
the degree of polymerization DP₁=(s₁+p₁+q₁) is less than or equal to 2,000, in particular less than 700, more particularly ranging from 40 to 450, in particular from 40 to 250, and in particular from 40 to 150,
the chain formation of the monomers of said general formula (I) can be random, monoblock or multiblock type.

According to a particularly preferred embodiment of the invention, the anionic polyelectrolyte of formula (I) has a mole fraction x_{P1} of monomers bearing hydrophobic groups Such that x_{P1}=p₁/(s₁+p₁+q₁) varies from 2 to 22%, in particular from 4 to 12% and even more particularly from 4 to 6%.

According to a particularly advantageous embodiment, the cationic polyelectrolyte according to the invention is of the following formula (II) or one of its pharmaceutically acceptable salts,

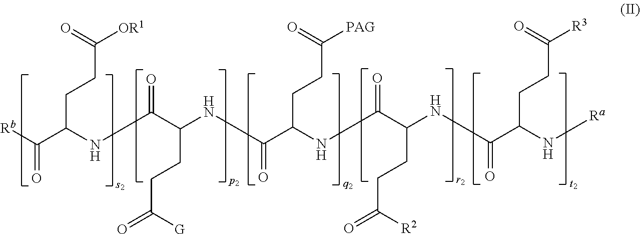

in which:
R^{a} represents a hydrogen atom, a linear C₂ to C₁₀ acyl group, a branched C₃ to C₁₀ acyl group, a pyroglutamate group or a hydrophobic group G as defined below;
R^{b} represents an —NHR⁵ group or a terminal amino acid residue bound by nitrogen and the carboxyl of which is optionally substituted by an —NHR⁵ alkylamino radical or an —OR⁶ alkoxy, in which:
   R⁵ represents a hydrogen atom, a linear C₁ to C₁₀ alkyl group, a branched C₃ to C₁₀ alkyl group, or a benzyl group;
   R⁶ represents a hydrogen atom, a linear C₁ to C₁₀ alkyl group, a branched C₃ to C₁₀ alkyl group, a benzyl group or a group G;
R¹ represents a hydrogen atom or a monovalent metal cation, preferably a sodium or potassium ion;
G represents a hydrophobic group chosen from: octyloxy-, dodecyloxy-, tetradecyloxy-, hexadecyloxy-, octadecyloxy-, 9-octadecenyloxy-, tocopheryl- and cholesteryl-;
PAG represents a polyalkylene glycol, preferably having a molar mass ranging from 1,800 to 6,000 g/mol, in particular a polyethylene glycol, in particular of molar mass ranging from 2,000 to 6,000 g/mol,
R² represents a cationic group, in particular arginine;
R³ represents a neutral group chosen from: hydroxyethylamino-, dihydroxypropylamino-;
   s₂ corresponds to the average number of non-grafted glutamate monomers, anionic at neutral pH,
   p₂ corresponds to the average number of glutamate monomers bearing a hydrophobic group G,
   q₂ corresponds to the average number of glutamate monomers bearing a polyalkylene glycol group,
   r₂ corresponds to the average number of glutamate monomers bearing a cationic group R²,
   t₂ corresponds to the average number of glutamate monomers bearing a neutral group R³,

s₂, p₂, q₂ and t₂ optionally being zero, and
the degree of polymerization DP₂=(s₂+p₂+q₂+r₂+t₂) is less than or equal to 2,000, in particular less than 700, more particularly varies from 40 to 450, in particular from 40 to 250, and in particular from 40 to 150;
the chain formation of the monomers of said general formula (II) can be random, monoblock or multiblock type.

Of course, the cationic polyelectrolyte corresponding to formula (II) is such that the overall charge of the polyelectrolyte (r₂−s₂) is positive.

According to a particularly preferred embodiment of the invention, the cationic polyelectrolyte of formula (II) has a mole fraction x_{P2} of monomers bearing hydrophobic groups such that x_{P2}=p₂/(s₂+p₂+q₂+r₂+t₂) varies from 2 to 22%, in particular from 4 to 12% and even more particularly from 4 to 6%.

Of course, said anionic polyelectrolyte and said cationic polyelectrolyte of the nanoparticles according to the invention, corresponding to the abovementioned formulae (I) and (II), are such that:
at least one of the two polyelectrolytes bears hydrophobic side groups G;
at least one of the two polyelectrolytes bears side groups of the polyalkylene glycol PAG type,

the quantity of said groups of the polyalkylene glycol type borne by the anionic and/or cationic polyelectrolyte being such that the mass ratio w_{PAG} of polyalkylene glycol relative to the total polymer is greater than or equal to 0.05, preferably comprised between 0.1 and 0.75, preferably between 0.15 and 0.6, preferably between 0.15 and 0.5, preferably between 0.15 and 0.3.

The mass ratio w_{PAG} after mixing the anionic and cationic polyelectrolytes can be calculated from the following formula:

$${w}_{PAG} = \frac{\begin{matrix}( {x}_{PAG 1} \cdot {m}_{1} \cdot {c}_{1} \cdot ( {DP}_{1} / {M}_{1} ) \cdot {M}_{PAG 1} ) + \\ ( {x}_{PAG 2} \cdot {m}_{2} \cdot {c}_{2} \cdot ( {DP}_{2} / {M}_{2} ) \cdot {M}_{PAG 2} )\end{matrix}}{( {m}_{1} \cdot {c}_{1} ) + ( {m}_{2} \cdot {c}_{2} )}$$

in which:
x_{PAG1} and x_{PAG2} represent the mole fractions of the monomers bearing polyalkylene glycol groups borne respectively by the anionic polyelectrolyte and the cationic polyelectrolyte,
M_{PAG1} and M_{PAG2} represent the molar masses of the polyalkylene glycol grafts borne respectively by the anionic polyelectrolyte and the cationic polyelectrolyte,

m₁ and m₂ respectively represent the mass quantities of the solutions before mixing the anionic polyelectrolyte and the cationic polyelectrolyte of respective mass concentrations of polymer (before mixing) C₁ and C₂;
DP₁ and DP₂ respectively represent the degrees of polymerization of the anionic polyelectrolyte and of the cationic polyelectrolyte;
M₁ and M₂ respectively represent the molar masses of the anionic polyelectrolyte and of the cationic polyelectrolyte.

According to a first embodiment, the anionic and cationic polyelectrolytes are such that:
the degree of polymerization of the anionic and cationic polyelectrolytes is comprised between 40 and 250, preferably between 40 and 110;
only one of the two polyelectrolytes bears hydrophobic side groups, distributed randomly;
only one of the two polyelectrolytes bears side groups of the polyalkylene glycol type, in particular polyethylene glycol groups of molar mass between 2,000 and 6,000 g/mol, distributed randomly;
t₂ is zero, i.e. the cationic polyelectrolyte is devoid of neutral groups;
the molar ratio Z of the number of cationic groups relative to the number of anionic groups in the mixture of the two polyelectrolytes is comprised between 0.1 and 2, preferably between 0.4 and 1.5.

According to a second embodiment, the anionic and cationic polyelectrolytes are such that:
the degree of polymerization of the anionic and cationic polyelectrolytes is comprised between 40 and 250, preferably between 40 and 110;
the anionic and cationic polyelectrolytes both bear hydrophobic side groups, distributed randomly;
only one of the two polyelectrolytes bears side groups of the polyalkylene glycol type, in particular polyethylene glycol groups of molar mass comprised between 2,000 and 6,000 g/mol, distributed randomly;
t₂ is zero, i.e. the cationic polyelectrolyte is devoid of neutral groups;
the molar ratio Z of the number of cationic groups relative to the number of anionic groups in the mixture of the two polyelectrolytes is comprised between 0.1 and 2, preferably between 0.4 and 1.5.

According to a third embodiment, the anionic and cationic polyelectrolytes are such that:
the degree of polymerization of the anionic and cationic polyelectrolytes is comprised between 40 and 250, preferably between 40 and 110;
the anionic and cationic polyelectrolytes both bear hydrophobic groups, distributed randomly;
the anionic and cationic polyelectrolytes both bear side groups of the polyalkylene glycol type, in particular polyethylene glycol groups of molar mass comprised between 2,000 and 6,000 g/mol, distributed randomly;
t₂ is zero, i.e. the cationic polyelectrolyte is devoid of neutral groups;
the molar ratio Z of the number of cationic groups relative to the number of anionic groups in the mixture of the two polyelectrolytes is comprised between 0.1 and 2, preferably between 0.4 and 1.5.

Examples of particularly preferred combinations of anionic and cationic polyelectrolytes according to the invention are described in the following variants.

According to a first preferred embodiment variant, the anionic and cationic polyelectrolytes are such that:
the mole fraction x_{P1} of hydrophobic groups in the anionic polyelectrolyte varies from 2 to 22%, in: particular from 4 to 12%;
the mole fraction x_{PAG1} of polyalkylene glycol groups in the anionic polyelectrolyte is zero;
the mole fraction x_{PAG2} of hydrophobic groups in the cationic polyelectrolyte is zero; and
the mole fraction x_{PAG2} of polyalkylene glycol groups in the cationic polyelectrolyte varies from 2 to 10%, in particular from 2 to 6%.

According to a second preferred embodiment variant, the anionic and cationic polyelectrolytes are such that:
the mole fraction x_{P1} varies from 2 to 22%, in particular from 4 to 12%;
the mole fraction x_{PAG1} varies from 2 to 10%, in particular from 2 to 6%;
the mole fraction x_{P2} is zero; and
the mole fraction x_{PAG2} is zero.

According to a third preferred embodiment variant, the anionic and cationic polyelectrolytes are such that:
the mole fraction x_{P1} varies from 2 to 22%, in particular from 4 to 12%;
the mole fraction x_{PAG1} varies from 2 to 10%, in particular from 2 to 6%;
the mole fraction x_{P2} varies from 5 to 20%, in particular from 5 to 10%; and
the mole fraction x_{PAG2} is zero.

According to a fourth preferred embodiment, the anionic and cationic polyelectrolytes are such that:
the mole fraction x_{P1} varies from 2 to 22%, in particular from 4 to 12%;
the mole fraction x_{PAG1} is zero;
the mole fraction x_{P2} varies from 5 to 20%, in particular from 5 to 10%; and
the mole fraction x_{PAG2} varies from 2 to 10%, in particular from 2 to 6%.

According to a fifth preferred embodiment variant, the anionic and cationic polyelectrolytes are such that:
the mole fraction x_{P1} varies from 2 to 22%, in particular from 4 to 12%;
the mole fraction x_{PAG1} varies from 2 to 10%, in particular from 2 to 6%;
the mole fraction x_{P2} varies from 5 to 20%; in particular from 5 to 10%; and
the mole fraction x_{PAG2} varies from 2 to 10%, in particular from 2 to 6%.

Nanoparticles

As previously stated, the nanoparticles formed according to the invention have an average diameter ranging from 10 to 100 nm.

Preferably, the size of the nanoparticles can vary from 10 to 70 nm, in particular from 10 to 50 nm.

The size of the nanoparticles can be measured by quasi-elastic light scattering.

Test for Measuring Particle Size by Quasi-Elastic Light Scattering

The particle size is characterized by the volume-average hydrodynamic diameter, obtained according to methods of measurement that are well known to a person skilled in the art, for example using a device of the ALV CGS-3 type.

Generally, the measurements are carried out with solutions of polymers prepared at concentrations of 1 mg/g in 0.15 M NaCl medium and stirred for 24 h. These solutions are then filtered on 0.8-0.2 μm, before being analysed by dynamic light scattering.

When using a device of the ALV CGS-3 type, operating with a vertically polarized He—Ne laser beam of wavelength 632.8 nm, the scattering angle is 140° and the signal acquisition time is 10 minutes. Measurement is repeated 3 times on two samples of solution. The result is the average of the 6 measurements.

Preparation of the Nanoparticles

The nanoparticles according to the invention can be obtained by mixing a solution of a first polyelectrolyte with a solution of a second polyelectrolyte of opposite polarity, said first and second polyelectrolytes being such that the mass ratio w_{PAG} of polyalkylene glycol relative to the total polymer is greater than or equal to 0.05.

The nanoparticles according to the invention can in particular be prepared according to the method comprising at least the stages consisting of:

(1) having an aqueous solution comprising nanoparticles of a first polyelectrolyte in the charged state, bearing hydrophobic side groups, said nanoparticles being non-covalently combined with an active ingredient;

(2) bringing said solution (1) together with at least one second polyelectrolyte of opposite polarity to that of the first polyelectrolyte,

with at least one of said first and second polyelectrolytes having/side groups of the polyalkylene glycol type, the quantity of said groups of the polyalkylene glycol type being such that the mass ratio w_{PAG} of polyalkylene glycol relative to the total polymer is greater than or equal to 0.05, preferably comprised between 0.1 and 0.75, preferably comprised between 0.15 and 0.6, preferably comprised between 0.15 and 0.5, preferably comprised between 0.15 and 0.3;

said first and second polyelectrolytes having a linear backbone of the polyamino acid type and having a degree of polymerization less than or equal to 2,000, preferably less than 700, in particular ranging from 40 to 450, in particular from 40 to 250, and in particular from 40 to 150.

In particular, said first and second polyelectrolytes are as defined previously.

According to a particular embodiment, the aqueous solution (1) is obtained by adding the active ingredient to an aqueous colloidal solution of the first polyelectrolyte, said active ingredient combining non-covalently with the nanoparticles of said first polyelectrolyte.

In particular, the aqueous solution (1) has a pH value ranging from 5 to 8, and more particularly of approximately 7.

According to a particular embodiment, stage (2) comprises at least:
the preparation of an aqueous solution of the second polyelectrolyte, in particular with a pH value ranging from 5 to 8, and advantageously with a pH value identical to that of the aqueous solution (1); and
the mixing of said aqueous solution of the second polyelectrolyte with said aqueous solution (1).

According to a particularly preferred embodiment, the first polyelectrolyte bears hydrophobic side groups, and is capable of spontaneously forming nanoparticles when it is dispersed in an aqueous medium with a pH ranging from 5 to 8, in particular water.

Without wishing to be bound by the theory it is possible to suggest that the supramolecular combination of the hydrophobic groups to form hydrophobic domains leads to the formation of nanoparticles. Each nanoparticle is thus constituted by one or more polyelectrolyte chains more or less condensed around these hydrophobic domains.

Preferably, the nanoparticles formed by the first polyelectrolyte, bearing hydrophobic side groups, have an average diameter ranging from 10 to 100 μm, in particular from 10 to 70 nm, and more particularly ranging from 10 to 50 nm.

The terms “combination” or “combined” used to describe the relationships between one or more active ingredients and the polyelectrolyte(s) mean that the active ingredient or ingredients are combined with the polyelectrolyte(s) by non-covalent physical interactions, in particular hydrophobic interactions, and/or electrostatic interactions and/or hydrogen bonds and/or via steric encapsulation by the polyelectrolytes.

According to another particular embodiment, the second polyelectrolyte also bears hydrophobic groups and is capable of forming nanoparticles when it is dispersed in an aqueous medium with a pH ranging from 5 to 8, in particular water.

The molar ratio, denoted Z, of the number of cationic groups relative to the number of anionic groups in the mixture of the two polyelectrolytes according to the invention is preferably comprised between 0.1 and 2, more particularly between 0.4 and 1.5.

The ratio Z reflects the overall charge of the nanoparticles and can, in particular, be close to zero, which may prove particularly interesting in certain applications.

According to a particularly advantageous embodiment of the invention, the molar ratio Z is comprised between 0.9 and 1.1, illustrating nanoparticles close to neutrality.

The molar ratio Z can be defined, with regard to the quantities and the nature of the polyelectrolytes introduced during the preparation of the nanoparticles according to the invention, by the following formula:

$$Z = \frac{( {x}_{c 2} \cdot {m}_{2} \cdot {C}_{2} \cdot {DP}_{2} / {M}_{2} )}{( {x}_{a 1} \cdot {m}_{1} \cdot {C}_{1} \cdot {DP}_{1} / {M}_{1} ) + ( {x}_{a 2} \cdot {m}_{2} \cdot {C}_{2} \cdot {DP}_{2} / {M}_{2} )}$$

in which:
m₁ and m₂ respectively represent the mass quantities of the solutions before mixing the anionic polyelectrolyte and the cationic polyelectrolyte of respective mass concentrations of polymer (before mixing) C₁ and C₂;
DP₁ and DP₂ respectively represent the degrees of polymerization of the anionic polyelectrolyte and of the cationic polyelectrolyte;
M₁ and M₂ respectively represent the molar masses of the anionic polyelectrolyte and of the cationic polyelectrolyte;
x_{c2} represents the mole fraction of monomers bearing cationic groups in the cationic polyelectrolyte;
xₐ₁ and xₙ₂ respectively represent the mole fractions of monomers bearing anionic groups of the anionic polyelectrolyte and of the cationic polyelectrolyte.

The nanoparticles can be anionic, cationic or neutral. Within the meaning of the invention, by “anionic nanoparticles” is meant nanoparticles the overall charge of which at neutral pH is negative; and by “cationic nanoparticles” is meant nanoparticles the overall charge of which at neutral pH is positive.

Moreover, according to another aspect of the invention, the nanoparticles according to the invention advantageously have a low overall electric charge, which generally makes it possible to improve the circulation time after intravenous administration.

The overall charge can be measured by any method known to a person skilled in the art, for example measurement of the Zeta potential at neutral pH.

Preferably, the nanoparticles according to the invention have a Zeta potential at neutral pH ranging from −20 mV to +20 mV, preferably ranging from −15 mV to +15 mV, preferably ranging from −10 mV to +10 mV.

Preferably, the solutions prepared in stages (1) and (2) have a total concentration of polyelectrolytes comprised between 1 and 50 mg/g, preferably between 5 and 50 mg/g, in particular from 7 to 25 mg/g.

Advantageously, the suspension of nanoparticles obtained at the end of stage (2) of the preparation method described above has a sufficient concentration of nanoparticles, and can be used as it is, without a further stage of concentration.

Advantageously, the suspension of nanoparticles obtained according to the method of the invention is suitable for administration by parenteral route, in particular by intravenous route.

Preferably, it has a viscosity, measured at 20° C. and at a shear rate of 10 s⁻¹, ranging from 1 to 500, preferably from 2 to 200 mPa·s.

The viscosity can be measured at 20° C., using standard equipment such as for example an imposed stress rheometer (Gemini, Bohlin) with geometry of the cone and plate type (4 cm and angle of 2°), or a Malvern Nanosizer viscometer, following the manufacturer's instructions.

According to another embodiment variant, the suspension of nanoparticles obtained at the end of stage (2) of the preparation method described above is subjected to one or more stages of concentration, in particular by tangential or frontal ultrafiltration, centrifugation, evaporation or lyophilization.

According to another embodiment variant, the method according to the invention can then comprise a stage of dehydrating the suspension of the obtained particles (for example by lyophilization or atomization), in order to obtain them in the form of dry powder.

Advantageously, the nanoparticles according to the invention are stable in the lyophilized form. Moreover, they are easy to redisperse after lyophilization. Thus, the suspension of nanoparticles according to the invention can be lyophilized then reconstituted in aqueous solution, without affecting the properties of the nanoparticles obtained.

The present invention also relates to novel pharmaceutical, phytosanitary, food, cosmetic or dietetic preparations made from the compositions according to the invention.

The composition according to the invention can thus be in the form of a powder, a solution, a suspension, a tablet or a gelatin capsule.

The composition of the invention can in: particular be intended for the preparation of a medicament.

It can be intended for administration by oral route or by parenteral route, in particular by parenteral route and more particularly by subcutaneous route.

The invention will be better explained by the following examples, given only by way of illustration.

### EXAMPLES

### Example 1

Synthesis of the Anionic Polyelectrolyte Polyglutamate Grafted with 5% of Vitamin E and with 4% of Polyethylene Glycol: with a Degree of Polymerization of Approximately 100

10 g of poly(glutamic acid) of DP 100 and 0.19 g of dimethylaminopyridine are solubilized in 160 mL of dimethylformamide (DMF) at 80° C. The mixture is stirred overnight at 80° C., cooled down to 15° C., then 1.67 g of α-tocopherol in solution in 6.5 mL of DMF, 0.285 g of dimethylaminopyridine, 16.2 g of polyethylene glycol (PEG) of mass 5,000 Da in solution in 32 mL of DMF and 1.8 mL of diisopropylcarbodiimide are added successively. The reaction mixture is stirred at 15° C. for 24 h, then neutralized with 1 N soda in a body of water. The solution obtained is purified by diafiltration and is concentrated.

The grafting rates with α-tocopherol and with polyethylene glycol, measured by proton NMR in TFA-d, are 5.3% and 3.8% respectively.

Table 1 below describes the characteristics of the anionic polyelectrolyte PA₁ (the notations p₁, q₁ and s₁ refer to formula (I) of the description; the notations x_{P1}, xₐ₁, x_{PAG1}, DP₁ are those defined in the description).

TABLE 1

| | | M₁ | | | | M_{PAG1} |
|---|---|---|---|---|---|---|
| Characteristics | DP₁ | (g/mol) | x_{P1} (%) | xₐ₁ (%) | x_{PAG1} (%) | (g/mol) |
| PA₁ | 100 | 37819 | 5 | 91 | 4 | 5229 |
| (p₁ = 5, q₁ = 4, | | | | | | |
| s₁ = 91) | | | | | | |

### Example 2

Synthesis of the Cationic Polyelectrolyte PC₁: Polyglutamate Grafted with 5% of Vitamin E and with 80% of Arginine, with a Degree of Polymerization of Approximately 100

The synthesis of this polymer is described in particular in the Applicant's International Application WO 2008/135563.

Table 2 below describes the characteristics of the cationic polyelectrolyte PC₁ (the notations p₂, q₂, r₂, s₂ and t₂ refer to formula (II) of the description; the notations DP₂, M₂, xₚ₂, xₐ₂, x_{c2}, x_{PAG2} and M_{PAG2} are those defined previously in the description).

TABLE 2

| | | M₂ | xₚ₂ | xₐ₂ | x_{c2} | x_{PAG2} | M_{PAG2} |
|---|---|---|---|---|---|---|---|
| Characteristics | DP₂ | (g/mol) | (%) | (%) | (%) | (%) | (g/mol) |
| PC₁ (p₂ = 5, q₂ = 0, | 100 | 30640 | 5 | 15 | 80 | 0 | — |
| r₂ = 80, s₂ = 15, t₂ = 0) | | | | | | | |

### Example 3

Preparation of Particles Based on the Two Polyelectrolytes PA₁ And PC₁ for Different Values of Z

The protocol for preparation of the polyelectrolyte complex is as follows:

The anionic polyelectrolyte PA₁ is diluted in a 10 mM solution of NaCl in order to obtain a mass m₁ of solution with the mass concentration C₁ while maintaining under moderate stirring. While still maintaining stirring, a quantity m₂ of a solution of the cationic polyelectrolyte PC₁, diluted beforehand to a mass concentration C₂ in a 10 mM solution of NaCl, is then poured into this solution.

The total mass concentration C of polyelectrolytes in the mixture obtained is given by the formula C=(m₁·C₁+m₂·C₂)/(m₁+m₂).

The diameter of the nanoparticles obtained is measured by quasi-elastic light scattering, as described previously.

The overall Zeta charge is measured by the measurement of the Zeta potential at neutral pH.

The values of the ratios Z (cationic groups/anionic groups molar ratio) and w_{PAG} (polyalkylene glycol/total polymer mass ratio), the total mass concentration C of polyelectrolytes in the mixture, the diameter, and the Zeta potential of the nanoparticles formed for different mixtures of solutions of the two polyelectrolytes PA₁ and PC₁ are shown in Table 3 below.

TABLE 3

| | | | | | | | | | Volume | |
|---|---|---|---|---|---|---|---|---|---|---|
| Tests | Polymers | m₁ (g) | C₁ (mg/g) | m₂ (g) | C₂ (mg/g) | C (mg/g) | Z | w_{PAG} | diameter (nm) | Zeta (mV) |
| e 1.1 | PA₁/PC₁ | 2.44 | 1.45 | 2.35 | 0.65 | 1.06 | 0.43 | 0.39 | 27 | −2 |
| e 1.2 | PA₁/PC₁ | 7.31 | 13.09 | 7.21 | 6.92 | 10.03 | 0.51 | 0.36 | 24 | −10 |
| e 1.3 | PA₁/PC₁ | 2.46 | 10.95 | 2.23 | 9.11 | 10.08 | 0.71 | 0.32 | 27 | −8 |
| e 1.4 | PA₁/PC₁ | 9.83 | 11.05 | 9.72 | 9.23 | 10.15 | 0.77 | 0.30 | 29 | −5 |
| e 1.5 | PA₁/PC₁ | 14.85 | 10.81 | 14.77 | 9.16 | 9.99 | 0.78 | 0.30 | 27 | −6 |
| e 1.6 | PA₁/PC₁ | 2.46 | 21.81 | 2.27 | 18.16 | 20.06 | 0.72 | 0.31 | 26 | −9.5 |
| e 1.7 | PA₁/PC₁ | 51.94 | 10.56 | 40.60 | 10.02 | 10.32 | 0.70 | 0.32 | 32 | −5 |

The results show that it is possible to obtain, from the mixture of anionic PA₁ and cationic PC₁ polyelectrolytes according to the invention, nanoparticles of a size less than or equal to 50 nm, in accordance with the invention.

### Example 4

### Comparative

Formulations Based on the Mixture of the Anionic Polyelectrolyte not Grafted by a Polyalkylene Glycol PA₂ (Polyglutamate Grafted with 5% of Vitamin E and with a Degree of Polymerization of Approximately 100, with Grafting of Polyethylene Glycol) and of the Cationic Polyelectrolyte PC₁ from Example 2

The synthesis of this polymer is in particular described in the Applicant's International Application WO 03/104303.

Table 4 below describes the characteristics of the non-PEGylated anionic polyelectrolyte PA₂ (the notations p₁, q₁ and s₁ refer to formula (I) of the description; the notations DP₁, M₁, xₚ₁, xₐ₁, x_{PAG1} and M_{PAG1} are those defined previously in the description).

TABLE 4

| | | M₁ | xₚ₁ | xₐ₁ | x_{PAG1} | M_{PAG1} |
|---|---|---|---|---|---|---|
| Characteristics | DP₁ | (g/mol) | (%) | (%) | (%) | (g/mol) |
| PA₂ (p₁= 5, q₁= 0, s₁ = 95) | 100 | 17064 | 5 | 95 | 0 | — |

In the same way as in Example 3, a quantity m₁ of a solution of the anionic polyelectrolyte PA₂ at concentration C₁ in a 10 mM solution of NaCl and a quantity m₂ of a solution of the cationic polyelectrolyte PC₁ described in Example 2, diluted beforehand to a concentration C₂ in a 10 mM solution of NaCl are prepared. In the same way as in Example 3, the cationic polymer PC₁ is added to the anionic polymer PA₂.

TABLE 5

| | | | | | | | | Volume | Zeta |
|---|---|---|---|---|---|---|---|---|---|
| Tests | Polymers | m₁ (g) | C₁ (mg/g) | m₂ (g) | C₂ (mg/g) | C (mg/g) | Z | diameter (nm) | (mV) |
| e 2.1 | PA₂/PC₁ | 2.40 | 1.32 | 2.70 | 2.01 | 1.69 | 0.70 | 150 | −55 |
| e 2.2 | PA₂/PC₁ | 2.40 | 11.06 | 3.52 | 12.96 | 12.19 | 0.70 | Flocculation | not |
| | | | | | | | | (>1 μm) | determined |

The results clearly show that nanoparticles obtained after mixing the polyelectrolytes not bearing polyalkylene glycol (w_{PAG}=0) are larger than 100 nm, not according to the invention.

### Example 5

Syntheses of Other Anionic Polyelectrolytes and of Cationic Polyelectrolytes Corresponding; to Formulae (I) and (II) According to the Invention

Synthesis of the Anionic Polyelectrolytes, PA₁ to PA₆:
PA₃ and PA₆ bear vitamin E grafts and polyethylene glycol groups. Their synthesis is similar to the synthesis of PA₁ proposed in Example 1.
PA₄ and PA₅ bear vitamin E grafts but are free from polyethylene glycol groups. The synthesis of such polymers is in particular described in the Applicant's International Application WO 03/104303.

Table 6 below summarizes the characteristics of the anionic polyelectrolytes that were prepared.

TABLE 6

| | | M₁ | xₚ₁ | xₐ₁ | x_{PAG1} | M_{PAG1} |
|---|---|---|---|---|---|---|
| | DP₁ | (g/mol) | (%) | (%) | (%) | (g/mol) |
| PA₃ (p₁ = 20, q₁ = 4, s₁ = 76) | 100 | 43680 | 20 | 76 | 4 | 5229 |
| PA₄ (p₁ = 11, q₁ = 0, s₁ = 209) | 220 | 37540 | 5 | 95 | 0 | — |
| PA₅ (p₁ = 10, q₁ = 0, s₁ = 90) | 100 | 19017 | 10 | 90 | 0 | — |
| PA₆ (p₁ = 5, q₁ = 2, s₁ = 93) | 100 | 37819 | 5 | 93 | 2 | 5229 |

Synthesis of the Cationic Polyelectrolytes, PC₁ to PC₈:
PC₂ bears vitamin E grafts but is free from polyethylene glycol groups and is free from neutral groups. Its synthesis, similar to the synthesis of PC₁ proposed in Example 2, is in particular described in the Applicant's International Application WO 2008/135563.
PC₆ bears vitamin E grafts, is free from polyethylene glycol groups and bears hydroxyethylamino-neutral groups. Its synthesis, similar to the synthesis of PC₂, in addition comprises a stage of grafting of ethanolamine. This grafting stage is described in the Applicant's International Application WO 2006/079614.
PC₇ bears vitamin E grafts and polyethylene glycol groups but is free from neutral groups. The synthesis of this polymer is as follows:

Stage 1: a polyglutamate grafted with 5% of vitamin E and with 4% of polyethylene glycol is synthesized following the protocol of Example 1.

Stage 2: the product from stage 1 is acidified to pH=3 and then lyophilized. 10 g of this lyophilizate is solubilized in 125 mL of NMP at 80° C. The solution obtained is cooled down to 0° C. and 3.15 mL of isobutyl chloroformate then 2.7 mL of N-methylmorpholine are added successively. The mixture is stirred for 15 min at 0° C.; a milky suspension is observed to form. In parallel, 8.36 g of argininamide dihydrochloride is suspended in 150 mL of NMP and 4.73 mL of triethylamine is added. The suspension obtained is stirred for a few minutes at 20° C. then cooled down to 0° C. The milky suspension of activated polymer is then added to this argininamide suspension, and the reaction mixture is stirred for 2 h at 0° C., then overnight at 20° C. After the addition of 2.4 mL of 1N HCl solution and then 2.5 mL of water, the reaction mixture is poured dropwise into 1.2 L of water. The solution obtained is purified by diafiltration and concentrated.

The percentage of argininamide grafted, determined by proton NMR in D₂O, is 84%.
PC₃ and PC₄ are free from vitamin E grafts, bear polyethylene glycol groups and hydroxyethylamino-neutral groups. The synthesis of this polymer is as follows:

10 g of poly(glutamic acid) of DP 100 is solubilized in 200 mL of NMP at 80° C. The solution obtained is cooled down to 0° C. and 10.5 mL of isobutyl chloroformate then 9 mL of N-methylmorpholine are added successively. The mixture is stirred for 15 min at 0° C. In parallel, 4.75 g of argininamide dihydrochloride is suspended in 94 mL of NMP and 2.3 mL of triethylamine is added. The suspension obtained is stirred for a few minutes at 20° C., then cooled down to 0° C. A solution of 5.46 g of polyethylene glycol (PEG) functionalized by a terminal amine, of mass 2,000 (MEPA-20H, sold by NOF) in 109 mL of NMP, then the argininamide/triethylamine suspension and 3.27 g of ethanolamine (EA) are added successively to the milky suspension of activated polymer. The reaction mixture is stirred overnight at 0° C. After the addition of 0.93 g of EA, the reaction mixture is stirred for 5 h at 20° C. After adding 0.77 mL of a 35% solution of HCl then 50 mL of water, the reaction mixture is poured dropwise into 500 mL of water and the pH is adjusted to 7-7.5 with 1N soda. The solution obtained is purified by diafiltration and concentrated. The percentages of PEG 2,000, of grafted EA and argininamide, determined by proton NMR in D₂O, are 3.70 and 25% respectively.
PC₅ is free from vitamin E grafts but bears polyethylene glycol groups and is free from neutral groups. Its synthesis is similar to the synthesis of PC₃ and PC₄ proposed above, except for the grafting of ethanolamine, which is not carried out for the synthesis of PC₅.
PC₈ is free from vitamin E grafts and polyethylene glycol groups but bears dihydroxypropylamino-neutral groups. The synthesis of this polymer is as follows:

Poly(glutamic acid) of DP 100 (62.8 g) is solubilized in 1,293 g of NMP at 80° C. The solution obtained is cooled down to 0° C. and 69.68 g of isobutyl chloroformate and then 51.6 g of N-methylmorpholine are added successively. The mixture is stirred for 15 min at 0° C. In parallel, 26.37 g of argininamide dihydrochloride is suspended in 501.98 g of NMP, and 33.2 g of aminopropanediol (APD) and then 10.79 g of triethylamine are added. The suspension obtained is stirred for a few minutes at 20° C., cooled down to 0° C., and then added to the milky suspension of activated polymer. The reaction mixture is stirred for 6 h at 0° C. 7.9 g of APD is then added, then the reaction mixture is stirred overnight at 0° C. After adding 52 g of 35% HCl solution, the reaction mixture is added dropwise to 5.4 L of water and the pH is adjusted to 7-7.5. The solution obtained is purified by diafiltration and is concentrated. The percentages of grafted APD and argininamide, determined by proton NMR in D₂O, are 72 and 18% respectively.

Table 7 below presents the characteristics of the cationic polymers prepared.

TABLE 7

| | | M₂ | xₚ₂ | xₐ₂ | x_{c2} | x_{PAG2} | M_{PAG2} |
|---|---|---|---|---|---|---|---|
| Characteristics | DP₂ | (g/mol) | (%) | (%) | (%) | (%) | (g/mol) |
| PC₂ (p₂ = 5, q₂ = 0, | 50 | 14600 | 10 | 30 | 60 | 0 | — |
| r₂ = 30, s₂ = 15, t₂ = 0) | | | | | | | |
| PC₃ (p₂ = 0, q₂ = 2, | 100 | 25901 | 0 | 8 | 20 | 2 | 3000 |
| r₂ = 20, s₂ = 8, t₂ = 70)^{(a)} | | | | | | | |
| PC₄ (p₂ = 0, q₂ = 3, | 100 | 27256 | 0 | 2 | 25 | 3 | 2182 |
| r₂ = 25, s₂ = 2, t₂ = 70)^{(a)} | | | | | | | |
| PC₅ (p₂ = 0, q₂ = 6, | 100 | 39841 | 0 | 24 | 70 | 6 | 2182 |
| r₂ = 70, s₂ = 24, t₂ = 0) | | | | | | | |
| PC₆ (p₂ = 10, q₂ = 0, | 100 | 26649 | 10 | 10 | 40 | 0 | — |
| r₂ = 40, s₂ = 10, t₂ = 40)^{(a)} | | | | | | | |
| PC₇ (p₂= 5, q₂ = 4, | 100 | 51396 | 5 | 11 | 80 | 4 | 5229 |
| r₂ = 80, s₂ = 11, t₂ = 0) | | | | | | | |
| PC₈ (p₂ = 0, q₂ = 0, | 100 | 22337 | 0 | 5 | 20 | 0 | — |
| r₂ = 20, s₂ = 5, t₂ = 75)^{(b)} | | | | | | | |

^{(a)}t₂ refers in this case to neutral grafts of the hydroxyethylamino-type
^{(b)}t₂ refers in this case to neutral grafts of the dihydroxypropylamino-type

### Example 6

Formulations Obtained from the Mixture of Anionic Polyelectrolytes PA and Cationic Polyelectrolytes PC Prepared in Example 5

The anionic polyelectrolyte PA is diluted in a 10 mM solution of NaCl in order to obtain a solution with the concentration C₁.

The cationic polyelectrolyte PC is diluted in a 10 mM solution of NaCl in order to obtain a solution with the concentration C₂.

The method then differs in the order of addition, depending on whether the final mixture sought has an excess of anionic charge or an excess of cationic charge:
for sought mixtures with an excess of anionic charge (test e 3.1 to e 3.9 in Table 8), a mass m₁ of anionic polyelectrolyte PA at concentration C₁ is placed in a beaker under moderate stirring and a mass m₂ of cationic polyelectrolyte PC at concentration C₂ is then added.
for sought mixtures with an excess of cationic charge (test e 3.10 in Table 8), a mass m₂ of cationic polyelectrolyte PC at concentration C₂ is placed in a beaker under moderate stirring and a mass m₁ of the anionic polymer PA at concentration C₁ is then added.

Table 8 below summarizes the masses and concentrations used in the mixtures as well as the characteristics of these mixtures.

TABLE 8

| | | | | | | | | | Volume | |
|---|---|---|---|---|---|---|---|---|---|---|
| Tests | Polymers | m₁ (g) | C₁ (mg/g) | m₂ (g) | C₂ (mg/g) | C (mg/g) | Z | w_{PAG} | diameter (nm) | Zeta (mV) |
| e 3.1 | PA₃/PC₁ | 14.80 | 12.47 | 14.85 | 7.50 | 9.98 | 0.77 | 0.30 | 29 | −4 |
| e 3.2 | PA₁/PC₂ | 12.37 | 6.52 | 7.32 | 15.94 | 10.02 | 0.76 | 0.23 | 34 | not |
| | | | | | | | | | | determined |
| e 3.3 | PA₁/PC₆ | 10.53 | 7.42 | 10.13 | 12.51 | 9.92 | 0.81 | 0.21 | 38 | not |
| | | | | | | | | | | determined |
| e 3.4 | PA₂/PC₃ | 10.00 | 2.91 | 10.00 | 17.37 | 10.14 | 0.62 | 0.20 | 11 | −6 |
| e 3.5 | PA₄/PC₄ | 1.31 | 1.90 | 0.22 | 71.90 | 11.97 | 0.97 | 0.21 | 23 | not |
| | | | | | | | | | | determined |
| e 3.6 | PA₂/PC₅ | 2.50 | 9.04 | 2.42 | 30.73 | 19.71 | 0.77 | 0.25 | 18 | −5 |
| e 3.7 | PA₂/PC₅ | 4.80 | 3.65 | 4.88 | 16.35 | 10.05 | 0.96 | 0.27 | 18 | 2 |
| e 3.8 | PA₂/PC₇ | 10.00 | 11.00 | 10.00 | 29.46 | 20.23 | 0.68 | 0.30 | 30 | −5 |
| e 3.9 | PA₁/PC₇ | 10.00 | 22.00 | 10.00 | 25.50 | 23.75 | 0.68 | 0.48 | 30 | −5 |
| e 3.10 | PA₂/PC₇ | 2.01 | 11.04 | 5.05 | 28.92 | 23.83 | 1.47 | 0.35 | 26 | 12 |

The results show that it is possible to obtain, from the mixture of the anionic and cationic polyelectrolytes according to the invention, nanoparticles smaller than 50 nm.

### Example 7

Formulations According to the Invention Incorporating Recombinant Human Growth Hormone (rhGH) as Active Ingredient

The rhGH is first mixed with the anionic polyelectrolyte PA and the PA/rhGH complex thus obtained is subsequently mixed with the cationic polyelectrolyte PC. More precisely:

The anionic polyelectrolyte PA is diluted in phosphate buffer to 20 mM and mixed with a solution containing 4.3 mg/g of rhGH (Biosides P161) so as to have a PA/rhGH mixture having a concentration C₁ of anionic polyelectrolyte PA and a concentration C_{P1} of rhGH protein. The mixture is left for 12 h at ambient temperature, under moderate stirring.

A mass m₁ of this PA/rhGH mixture is added to a mass m₂ of cationic polyelectrolyte at concentration C₂. The final mixture has a total polymer concentration C (calculated as in Example 3) and a protein concentration Cₚ=m₁Cₚ₁/(m₁+m₂).

The concentration of active ingredient not combined with the polyelectrolytes is determined by analysing the final mixture by steric exclusion chromatography (columns G4,000+G2,000 SWXL—PBS buffer diluted one tenth—flow 0.5 mL/min). In all cases, the peak corresponding to the elution time of non-combined rhGH is not detected: the fraction of non-combined rhGH is therefore <1%.

Table 9 below presents the masses and concentrations used in the mixture as well as the characteristics of this mixture.

TABLE 9

| | | | C₁ | Cₚ₁ (mg/g) | | C₂ | Cₚ (mg/g) | C | | | Volume | Zeta |
|---|---|---|---|---|---|---|---|---|---|---|---|---|
| Tests | Polymers | m₁ (g) | (mg/g) | (rhGH) | m₂ (g) | (mg/g) | (rhGH) | (mg/g) | Z | w_{PAG} | diameter (nm) | (mV) |
| e 4.1 | PA₆/PC₂ | 10.15 | 5.00 | 0.28 | 18.26 | 5.01 | 0.10 | 5.01 | 0.71 | 0.14 | 22 | −7 |
| c 4.2 | PA₆/PC₂ | 20.21 | 5.03 | 0.27 | 18.26 | 5.02 | 0.14 | 5.03 | 0.43 | 0.20 | 43 | −6 |
| e 4.3 | PA₆/PC₈ | 8.70 | 3.02 | 0.34 | 21.42 | 5.90 | 0.10 | 5.07 | 0.96 | 0.07 | 55 | −2 |

The results show that the formulations according to the invention incorporating rhGH protein and polyelectrolytes according to the invention are composed of nanoparticles smaller than 50 nm.

### Example 8

Formulations According to the Invention Incorporating, as Active Ingredient, Recombinant Human Growth Hormone (rhGH), Concentrated by Two Methods: Ultrafiltration and Lyophilization/Reconstitution

A fraction of the formulation described in test e 4.2 of Example 7 (with the polyelectrolytes PA₆ and PC₂) is lyophilized using a benchtop lyophilizer (CHRIST Alpha 2-4 LP plus) for 24 hours. The lyophilized powder is then dispersed in water so as to obtain a solution approximately 20 times more concentrated than the solution in the previous test e 4.2. A homogeneous colloidal solution is obtained in less than 5 minutes.

Another fraction of the formulation is concentrated by a factor of approximately 10 by frontal ultrafiltration on a membrane having a cutoff of 10 kDa.

The characteristics of the solutions before and after concentration are presented in Table 10 below.

TABLE 10

| | Concen- | Total concen- | Volume | Concen- |
|---|---|---|---|---|
| | tration | tration of | diam- | tration of |
| | of rHGH | polyelectrolytes | eter | free rhGH |
| | (mg/g) | (mg/g) | (nm) | (%) |
| Before concentration | 0.14 | 5.03 | 43 | <5 |
| (test e 4.2) | | | | |
| Lyophilization/ | 1.56 | 54.31 | 44 | <5 |
| reconstitution | | | | |
| Concentration by | 1.37 | 47.81 | 43 | <5 |
| ultrafiltration | | | | |

The results clearly show that the formulations according to the invention can easily be concentrated without changing the size of the particles.

### Example 9

Formulations According to the Invention Incorporating Salmon Calcitonin (sCT) as Active Ingredient

The sCT is firstly mixed with the anionic polyelectrolyte PA and the PA/sCT complex thus obtained is subsequently mixed with the cationic polyelectrolyte PC. More precisely:

The anionic polyelectrolyte PA is diluted in a 10 mM solution of phosphate buffer and mixed with a solution containing 10 mg/g of sCT (Polypeptide Laboratories AB) so as to obtain a PA/sCT mixture having a concentration C₁ of anionic polyelectrolyte PA and a concentration Cₚ of protein sCT. The mixture is stirred for 1 h at ambient temperature with a magnetic bar.

The method then differs in the order of addition, depending on whether the sought final mixture has an excess of anionic charge or an excess of cationic charge:
for sought mixtures with an excess of anionic charge (tests e 5.1 and e 5.2 in the table given below), a mass m₁ of the previous mixture PA/sCT is placed in a beaker under moderate stirring and a mass m₂ of cationic polyelectrolyte PC, diluted beforehand to concentration C₂ is then added to the mixture;
for sought mixtures with an excess of cationic charge (test e 5.3 in the table given below), a mass m₂ of cationic polyelectrolyte PC, diluted beforehand to concentration C₂ is placed in a beaker under moderate stirring and a mass m₁ of the previous mixture PA/sCT is then added to the mixture.

The final mixture has a total polymer concentration C and a protein concentration Cₚ.

The concentration of active ingredient not combined with the polyelectrolytes is determined after separation by ultracentrifugation on ultrafilters having a cutoff of 30 kDa and assay of the filtrates by HPLC. In all cases it is strictly less than 5%.

The characteristics of the anionic and cationic polyelectrolytes used for this example are described in Example 5.

TABLE 11

| | | | | Cₚ₁ (mg/g) | | C₂ | Cₚ (mg/g) | C | | | Volume | Zeta |
|---|---|---|---|---|---|---|---|---|---|---|---|---|
| Tests | Polymers | m₁ (g) | C₁ (mg/g) | (sCT) | m₂ (g) | (mg/g) | (sCT) | (mg/g) | Z | w_{PAG} | diameter (nm) | (mV) |
| e 5.1 | PA₄/PC₇ | 15.01 | 0.91 | 0.91 | 3.59 | 8.00 | 0.74 | 2.28 | 0.54 | 0.30 | 32 | −7 |
| e 5.2 | PA₄/PC₇ | 14.93 | 0.95 | 0.48 | 3.73 | 7.97 | 0.38 | 2.35 | 0.60 | 0.12 | 29 | −11 |
| e 5.3 | PA₅/PC₄ | 25.02 | 4.00 | 0.33 | 25.01 | 31.99 | 0.17 | 17.99 | 1.4 | 0.37 | 16 | −3 |

The results show that the formulations according to the invention incorporating salmon calcitonin and polyelectrolytes according to the invention are composed of nanoparticles smaller than 50 nm.

### Example 10

Formulations According to the Invention Incorporating Recombinant Human Insulin (INS) as Active Ingredient

The insulin is firstly mixed with the anionic polyelectrolyte PA and the PA/INS complex thus obtained is subsequently mixed with the cationic polyelectrolyte PC. More precisely:

A stock solution of INS (Biocon) is prepared as follows:

0.36 g of INS is dissolved in 9 g of distilled water, the solution is stirred for 10 minutes at 250 rpm with a magnetic bar. The solution is acidified by adding 2.66 g of a 0.1 N solution of hydrochloric acid, and then stirred at 500 rpm for 15 minutes. 3.98 g of a 0.1 N solution of sodium hydroxide is then added while stirring at 500 rpm, then after 15 minutes, 3.90 g of distilled water is added, to obtain a final concentration of INS of 17.54 mg/g (taking into account the percentage of water present in the insulin powder).

The anionic polyelectrolyte PA is diluted in a 10 mM solution of sodium chloride and mixed with the insulin stock solution containing 17.54 mg/g of INS (Biocon) so as to obtain a PA/sCT mixture having a concentration C₁ of anionic polyelectrolyte PA and a concentration Cₚ of protein INS. The mixture is stirred moderately for 20 h at ambient temperature.

A mass m₂ of cationic polyelectrolyte, diluted beforehand to the concentration C₂ with a 10 mM solution of NaCl, is added, under stirring, to a mass m₁ of this PA/INS mixture in a beaker. The final mixture has a total polymer concentration C (calculated as previously) and a concentration of protein Cp (calculate as previously).

The concentration of active ingredient not combined with the polyelectrolytes is determined after separation by ultracentrifugation on ultrafilters having a cutoff of 50 kDa and assay of the filtrates by HPLC. The concentration of non-combined active ingredient under these conditions is approximately 8%. The anionic and cationic polyelectrolytes used for this example are the polyelectrolytes PA₄ and PC₇ respectively, described in Example 5.

TABLE 12

| | | m₁ | C₁ | Cₚ₁ (mg/g) | m₂ | C₂ | Cₚ (mg/g) | C | | | Volume | Zeta |
|---|---|---|---|---|---|---|---|---|---|---|---|---|
| Tests | Polymers | (g) | (mg/g) | (INS) | (g) | (mg/g) | (INS) | (mg/g) | Z | w_{PAG} | diameter (nm) | (mV) |
| e 6.1 | PA₄/PC₇ | 4 | 4.99 | 1.02 | 2 | 18.81 | 0.68 | 9.6 | 0.7 | 0.27 | 48 | −5 |

The results show that the formulations according to the invention incorporating recombinant human insulin and polyelectrolytes according to the invention are composed of nanoparticles smaller than 50 nm.

### Example 11

Measurements of the Viscosity of the Formulations According to the Invention

A fraction of the formulation described in test e 1.7 of Example 3 (with the polyelectrolytes PA₁ and PC₁) is lyophilized using a benchtop lyophilizer (CHRIST Alpha 2-4 LP plus). The lyophilized powder is then dispersed in water to obtain a solution approximately 10 times more concentrated than the solution in test e 1.7. A homogeneous colloidal solution is obtained in less than 5 minutes.

The viscosity is measured at 20° C., at a shear rate of 10 s⁻¹, using an imposed stress rheometer (Gemini, Bohlin) with geometry of the cone and plate type (4 cm and angle of 2°).

TABLE 13

| Total concentration of | Viscosity |
|---|---|
| polyelectrolytes (mg/g) | (mPa · s) |
| 95.8 | 18 |
| 81.2 | 4 |
| 59.4 | 5 |
| 40.8 | 2 |
| 23.7 | 1 |
| 10.3 | 1 |
| 5 | 1 |

The results show that the formulations according to the invention are sufficiently fluid for injection by parenteral route, and in particular by subcutaneous route.

1. Nanoparticle formed by at least one active ingredient and by at least two polyelectrolytes of opposite polarity having a linear backbone of the polyamino acid type and having a degree of polymerization less than or equal to 2,000, characterized in that:
at least one of the two polyelectrolytes bears hydrophobic side groups;
at least one of the two polyelectrolytes bears side groups of the polyalkylene glycol type;
said nanoparticles having an average diameter ranging from 10 to 100 nm and comprising a quantity of groups of the polyalkylene glycol type such that the mass ratio w_{PAG} of polyalkylene glycol relative to the total polymer is greater than or equal to 0.05.

2. Nanoparticle according to claim 1, characterized in that it is obtained by mixing a solution of a first polyelectrolyte with a solution of a second polyelectrolyte of opposite polarity, said first and second polyelectrolytes being such that the mass ratio w_{PAG} of polyalkylene glycol relative to the total polymer is greater than or equal to 0.05.

3. Nanoparticle according to any one of the previous claims, characterized in that the molar ratio Z of the number of cationic groups relative to the number of anionic groups borne by the anionic and cationic polyelectrolytes used is comprised between 0.1 and 2, more particularly between 0.4 and 1.5.

4. Nanoparticle according to any one of the previous claims, characterized in that the mass ratio w_{PAG} of polyalkylene glycol relative to the total polymer ranges from 0.1 to 0.75, in particular from 0.15 to 0.6, in particular from 0.15 to 0.5 and preferably from 0.15 to 0.3.

5. Nanoparticle according to any one of the previous claims, characterized in that the size of the nanoparticles varies from 10 to 70 nm, preferably from 10 to 50 nm.

6. Nanoparticle according to any one of the previous claims, characterized in that said polyelectrolyte bearing hydrophobic side groups is capable of spontaneously forming nanoparticles when it is dispersed in an aqueous medium with a pH ranging from 5 to 8, in particular water.

7. Nanoparticle according to any one of the previous claims, characterized in that said anionic polyelectrolyte is of the following formula (I) or a pharmaceutically acceptable salt thereof, 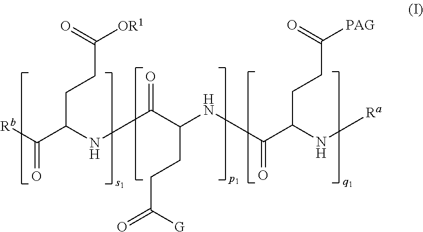
in which:
R^{a} represents a hydrogen atom, a linear C₂ to C₁₀ acyl group, a branched C₃ to C₁₀ acyl group, a pyroglutamate group or a hydrophobic group G as defined below;
R^{b} represents an —NHR⁵ group or a terminal amino acid residue bound by nitrogen and the carboxyl of which is optionally substituted by an —NHR⁵ alkylamino radical or an —OR⁶ alkoxy, in which:
R⁵ represents a hydrogen atom, a linear C₁ to C₁₀ alkyl group, a branched C₃ to C₁₀ alkyl group, or a benzyl group;
a R⁶ represents a hydrogen atom, a linear C₁ to C₁₀ alkyl group, a branched C₃ to C₁₀ alkyl group, a benzyl group or a group G;
R¹ represents a hydrogen atom or a monovalent metal cation, preferably a sodium or potassium ion,
G represents a hydrophobic group chosen from: octyloxy-, dodecyloxy-, tetradecyloxy-, hexadecyloxy-, octadecyloxy-, 9-octadecenyloxy-, tocopheryl- and cholesteryl-;
PAG represents a polyalkylene glycol, preferably having a molar mass ranging from 1,800 to 6,000 g/mol, in particular a polyethylene glycol, in particular of molar mass ranging from 2,000 to 6,000 g/mol,
s₁ corresponds to the average number of non-grafted glutamate monomers, anionic at neutral pH,
p₁ corresponds to the average number of glutamate monomers bearing a hydrophobic group G, and
q₁ corresponds to the average number of glutamate monomers bearing a polyalkylene glycol group,
p₁ and q₁ optionally being zero,
the degree of polymerization DP₁=(s₁+p₁+q₁) is less than or equal to 2,000, in particular less than 700, more particularly ranging from 40 to 450, in particular from 40 to 250, and in particular from 40 to 150,
the chain formation of the monomers of said general formula (I) can be random, monoblock or multiblock type.

8. Nanoparticle according to any one of the previous claims, characterized in that said cationic polyelectrolyte is of the following formula (II) or a pharmaceutically acceptable salt thereof, 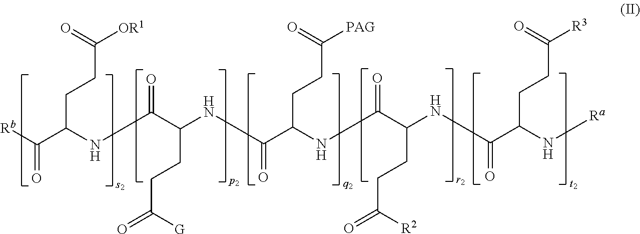
in which:
R^{a} represents a hydrogen atom, a linear C₂ to C₁₀ acyl group, a branched C₃ to C₁₀ acyl group, a pyroglutamate group or a hydrophobic group G as defined below;
R^{b} represents an —NHR⁵ group or a terminal amino acid residue bound by nitrogen and the carboxyl of which is optionally substituted by an —NHR⁵ alkylamino radical or an —OR⁶ alkoxy, in which:
R⁵ represents a hydrogen atom, a linear C₁ to C₁₀ alkyl group, a branched C₃ to C₁₀ alkyl group, or a benzyl group;
R⁶ represents a hydrogen atom, a linear C₁ to C₁₀ alkyl group, a branched C₃ to C₁₀ alkyl group, a benzyl group or a group G;
R¹ represents a hydrogen atom or a monovalent metal cation, preferably a sodium or potassium ion;
G represents a hydrophobic group chosen from: octyloxy-, dodecyloxy-, tetradecyloxy-, hexadecyloxy-, octadecyloxy-, 9-octadecenyloxy-, tocopheryl- and cholesteryl-;
PAG represents a polyalkylene glycol, preferably having a molar mass ranging from 1,800 to 6,000 g/mol, in particular a polyethylene glycol, in particular of molar mass ranging from 2,000 to 6,000 g/mol,
R² represents a cationic group, in particular arginine;
R³ represents a neutral group chosen from: hydroxyethylamino-, dihydroxypropylamino-;
s₂ corresponds to the average number of non-grafted glutamate monomers, anionic at neutral pH,
p₂ corresponds to the average number of glutamate monomers bearing a hydrophobic group G,
q₂ corresponds to the average number of glutamate monomers bearing a polyalkylene glycol group,
r₂ corresponds to the average number of glutamate monomer's bearing a cationic group R²,
t₂ corresponds to the average number of glutamate monomers bearing a neutral group R³,
s₂, p₂, q₂ and t₂ optionally being zero, and
the degree of polymerization DP₂=(s₂+p₂+q₂+r₂+t₂) is less than or equal to 2,000, in particular less than 700, more particularly varies from 40 to 450, in particular from 40 to 250, and in particular from 40 to 150;
the chain formation of the monomers of said general formula (II) can be random, monoblock or multiblock type.

9. Nanoparticle according to any one of the previous claims, characterized in that the anionic and cationic polyelectrolytes are such that:
the mole fraction x_{P1} of hydrophobic groups in the anionic polyelectrolyte varies from 2 to 22%, in particular from 4 to 12%;
the mole fraction x_{PAG1} of polyalkylene glycol groups in the anionic polyelectrolyte is zero;
the mole fraction x_{P2} of hydrophobic groups in the cationic polyelectrolyte is zero; and
the mole fraction x_{PAG2} of polyalkylene glycol groups in the cationic polyelectrolyte varies from 2 to 10%, in particular from 2 to 6%.

10. Nanoparticle according to any one of claims 1 to 8, characterized in that the anionic and cationic polyelectrolytes are such that:
the mole fraction x_{P1} varies from 2 to 22%, in particular from 4 to 12%;
the mole fraction x_{PAG1} varies from 2 to 10%, in particular from 2 to 6%;
the mole fraction x_{P2} is zero; and
the mole fraction x_{PAG2} is zero.

11. Nanoparticle according to any one of claims 1 to 8, characterized in that the anionic and cationic polyelectrolytes are such that:
the mole fraction x_{P1} varies from 2 to 22%, in particular from 4 to 12%;
the mole fraction x_{PAG1} varies from 2 to 10%, in particular from 2 to 6%;
the mole fraction x_{P2} varies from 5 to 20%, in particular from 5 to 10%; and
the mole fraction x_{PAG2} is zero.

12. Nanoparticle according to any one of claims 1 to 8, characterized in that the anionic and cationic polyelectrolytes are such that:
the mole fraction x_{P1} varies from 2 to 22%, in particular from 4 to 12%;
the mole fraction x_{PAG1} is zero;
the mole fraction x_{P2} varies from 5 to 20%, in particular from 5 to 10%; and
the mole fraction x_{PAG2} varies from 2 to 10%, in particular from 2 to 6%.

13. Nanoparticle according to any one of claims 1 to 8, characterized in that the anionic and cationic polyelectrolytes are such that:
the mole fraction x_{P1} varies from 2 to 22%, in particular from 4 to 12%;
the mole fraction x_{PAG1} varies from 2 to 10%, in particular from 2 to 6%;
the mole fraction x_{P2} varies from 5 to 20%, in particular from 5 to 10%; and
the mole fraction x_{PAG2} varies from 2 to 10%, in particular from 2 to 6%.

14. Nanoparticle according to any one of the previous claims, characterized in that said active ingredient is a molecule of therapeutic, cosmetic or prophylactic interest or of interest for imaging.

15. Composition, characterized in that it comprises at least nanoparticles as defined according to any one of the previous claims.

16. Method for the preparation of nanoparticles as defined according to any one of claims 1 to 14, characterized in that it comprises at least the stages consisting of:
(1) having an aqueous solution comprising nanoparticles of a first polyelectrolyte in the charged state, bearing hydrophobic side groups, said nanoparticles being non-covalently combined with an active ingredient;
(2) bringing said solution (1) together with at least one second polyelectrolyte of opposite polarity to that of the first polyelectrolyte, so as to form said nanoparticles,
with at least one of said first and second polyelectrolytes having side groups of the polyalkylene glycol type, the quantity of said groups of the polyalkylene glycol type being such that the mass ratio w_{PAG} of polyalkylene glycol relative to the total polymer is greater than or equal to 0.05;
said first and second polyelectrolytes having a linear backbone of the polyamino acid type and having a degree of polymerization less than or equal to 2,000.

17. Method according to the previous claim, characterized in that said first and second polyelectrolytes are as defined according to any one of claims 3, 4 and 6 to 13.

18. Method according to either of claims 16 and 17, characterized in that the aqueous solution (1) is obtained by adding the active ingredient to an aqueous colloidal solution of the first polyelectrolyte, in particular having a pH value ranging from 5 to 8, said active ingredient combining non-covalently with the nanoparticles of said first polyelectrolyte.

19. Method according to any one of claims 16 to 18, characterized in that stage (2) comprises at least:
the preparation of an aqueous solution of the second polyelectrolyte, in particular with a pH value ranging from 5 to 8, and advantageously with a pH value identical to that of the aqueous solution of stage (1); and
the mixing of said aqueous solution of the second polyelectrolyte with said aqueous solution of stage (1).
